(19) United States
(12) Patent Application Publication
(10) Pub. No.: US 2014/0272948 A1
(43) Pub. Date: Sep. 18, 2014

(54) HIERARCHICAL ASSEMBLY OF OPTICAL MAPS

(71) Applicant: OpGen, Inc., Gaithersburg, MD (US)

(72) Inventor: Deacon John Sweeney, Gaithersburg, MD (US)

(73) Assignee: OpGen, Inc., Gaithersburg, MD (US)

(21) Appl. No.: 14/212,581

(22) Filed: Mar. 14, 2014

Related U.S. Application Data
(60) Provisional application No. 61/790,811, filed on Mar. 15, 2013.

(51) Int. Cl. C12Q 1/68

(52) U.S. Cl. CPC C12Q 1/68
USPC 435/6.1

(57) ABSTRACT

The invention generally relates to optical maps and particularly to computationally tractable methods of assembling large numbers of single molecule maps by dividing the maps into smaller groups of maps within which all of the maps are similar to one another by some metric. For each group, all of the maps are assembled into contigs. The resulting contigs are then assembled into one or more genome assemblies. By dividing the maps into groups, a number of comparison operations required for assembly is reduced and, since each group of maps can be assembled into a contig in a discrete operation, the overall assembly operation can be parallelized.

### RELATED APPLICATION

The present application claims the benefit of and priority to U.S. provisional application Ser. No. 61/790,811, filed Mar. 15, 2013, which is incorporated by reference herein in its entirety.

### FIELD OF THE INVENTION

The invention generally relates to optical maps and particularly to computationally tractable methods of assembling large numbers of single molecule maps.

### BACKGROUND

A physical map of a genome can provide accurate information about the organization of a chromosome and can be valuable when genomes are sequenced. In fact, some sequencing results can only be properly assembled through the use of a physical map. Attempts to create physical maps have used sequence tagged sites, hybridization, and restriction mapping. Optical mapping is a method of physical mapping that produces ordered maps of restriction fragments and sites by using microscopy to study restriction sites on individual molecules of nucleic acid. Optical maps provide details relating to chromosomal structures and abnormalities such as insertions, deletions, repetitive sequences, palindromes, translocations, duplications, inversions, or others.

Optical mapping has been used to study certain smaller genomes. However, available data processing algorithms make it impractical to assemble whole genome optical maps for large genomes such as a human genome. While front-end data collection techniques are available for covering a whole genome with single molecule optical maps, to assemble such a set of single molecule optical maps into a whole genome optical map by existing data processing algorithms would require months or longer. Additionally, existing assembly algorithms may not be adequate for assembling maps from mixed or heterozygous samples. For example, assembling a set of single molecule optical maps from a diploid organism or from a mixed sample of closely-related organisms by known algorithms may lead to a number of false matches between individual maps that are hard to detect and hard to correct.

### SUMMARY

The invention provides methods of assembling a plurality of single molecule optical maps by dividing the maps into smaller groups of maps within which all of the maps are similar to one another by some metric. For each group, all of the maps are assembled into contigs. The resulting contigs are then assembled into one or more genome assemblies, each representing a chromosome or genome from the starting sample. By dividing the maps into groups, a number of comparison operations required for assembly is reduced drastically (for example, pairwise comparisons among 100 maps requires 9.3×10¹⁵⁷ comparison operations whereas pairwise comparisons among 10 groups of 10 maps requires 10×3.6×10⁶, or 3.6×10⁷, operations). Additionally, since each group of maps can be assembled into a contig in a discrete operation, the overall assembly operation can be parallelized with ease. Grouping the single molecule optical maps by the similarity metric also provides for the segregation of maps into groups by source chromosome. Where the starting sample is heterozygous, this will produce haplotype-specific contigs. This provides for a novel method of mapping the heterozygous information: the contigs can be linked together in a branched path in which convergent sections of the path represent regions of similarity and divergent regions of the path represent dissimilarity.

Since a large number of single molecule optical maps can be assembled rapidly, and optionally in parallel, whole genome optical map assemblies can be produced in practicable times using widely available computing resources. Thus, whole genomes, mixed samples, and heterozygous samples can all be rapidly mapped completely and accurately and represented by a branched path display. This gives the ability to study a history of chromosome rearrangements or perform detailed comparative analyses of chromosomal structure. Additionally, the whole genome optical maps can provide the necessary physical maps to aid in assembly of genome sequences.

In certain aspects, the invention provides a method of obtaining genomic information that includes generating a plurality of single molecule optical maps from a nucleic acid sample, linking pairs of the maps according to a similarity metric, dividing the plurality of maps into groups comprising linked maps, assembling the linked maps within each group into a contig to produce a plurality of contigs, and assembling the plurality of contigs into at least one genome assembly. Optionally, the method can be iterative and include linking pairs of the contigs according to a contig similarity metric, dividing the contigs into contig groups comprising linked contigs, assembling the linked contigs within each contig group into a scaffold to produce a plurality of scaffolds and assembling the plurality of scaffolds into the at least one genome assembly. The plurality of contigs may be assembled into two or more genome assemblies.

The similarity metric can include a Chen-Stein probability distribution comparison, a measure of a number of cutting sites per length of nucleic acid molecule for a restriction enzyme, or some other measure of similarity. For example, the method may include generating an in silico ordered restriction map from a reference genome and obtaining the similarity metric by comparing the maps to the in silico ordered restriction

In some embodiments, the invention provides a heterozygosity display and methods of assembling the plurality of contigs comprising linking the plurality of contigs together to form a branched path. The branched path may include at least converged section and a diverged section. Preferably, the at least one diverged section represents heterozygosity in the sample. In this way, the genome assembly represents a diploid genome that is heterozygous at the diverged section.

In certain embodiments, generating a single molecule optical map includes introducing nucleic acid from the sample to a charged substrate so that the nucleic acids become elongated and fixed on the substrate in a manner in which the nucleic acids remain accessible for enzymatic reactions, digesting the nucleic acids enzymatically to produce one or more restriction digests, and constructing a map from the restriction digests. The substrate may be derivatized glass. A sample can be human tissue or fluid, or an environmental sample, or some other type of sample.

Aspects of the invention provide a method of obtaining contigs that includes generating a plurality of single-molecule ordered restriction maps from a nucleic acid sample, segregating the maps into a plurality of groups, each group comprising a plurality of linked maps linked by a similarity metric, and assembling the linked maps within each group into a contig to produce a plurality of contigs. The method may optionally further include linking pairs of the contigs that satisfy a contig similarity metric, segregating the contigs into contig groups comprising linked contigs, and assembling the linked contigs within each contig group into a scaffold to produce a plurality of scaffolds. In some embodiments, the method includes assembling the plurality of scaffolds into the at least one genome assembly.

### BRIEF DESCRIPTION OF THE DRAWINGS

FIG. 1 diagrams a method of optical mapping.

FIG. 2 is a flow chart of methods of map assembly.

FIG. 3 shows a system of the invention.

FIG. 4 illustrates a display to represent heterozygosity.

### DETAILED DESCRIPTION

The invention generally relates to a hierarchical approach to assembling single-molecule ordered restriction maps such as optical maps into contigs.

In general, single molecule optical maps are created and are then separated or segregated by similarity. Any method of grouping single molecule ordered restriction maps by similarity may be used such as, for example, generating tables of links between molecules based on a similarity metric. Groups of closely related maps are made. For each group, the single molecule optical maps are processed to produce contigs. Those contigs can be assembled into whole genome maps or segregated themselves into groups of contigs, grouped by similarity.

Each of the following sections addresses considerations for one of a variety of topics relevant to embodiments of the invention, including sample extraction, optical mapping, and assembly of optical maps.

### Sample Nucleic Acids

Nucleic acids include deoxyribonucleic acid (DNA), ribonucleic acid (RNA), or both. Nucleic acids can be synthetic or derived from naturally occurring sources. In one embodiment, nucleic acids are isolated from a biological sample containing a variety of other components, such as proteins, lipids and non-sample nucleic acids. Nucleic acids can be obtained from any cellular material, obtained from a human or other mammal, plant, or microorganism (e.g., bacterium, fungus, virus or any other cellular organism). In certain embodiments, the nucleic acids are obtained from a single cell. Biological samples for use in the present invention include viral particles or preparations. Nucleic acids can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acids can also be isolated from cultured cells, such as a primary cell culture or a cell line. The cells or tissues from which nucleic acids are obtained can be infected with a virus or other intracellular pathogen. A sample can also be total RNA extracted from a biological specimen, a cDNA library, viral, or genomic DNA.

Nucleic acids obtained from biological samples typically are fragmented to produce suitable fragments for analysis. In one embodiment, nucleic acid from a biological sample is fragmented by sonication. Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, N.Y. 2,028 pages (2012); or as described in U.S. Pat. No. 7,957,913; U.S. Pat. No. 7,776,616; U.S. Pat. No. 5,234,809; U.S. Pub. 2010/0285578; and U.S. Pub. 2002/0190663. Nucleic acid molecules may be single-stranded, double-stranded, or double-stranded with single-stranded regions (for example, stem- and loop-structures).

Nucleic acids obtained from biological samples may be fragmented to produce suitable fragments for analysis. Template nucleic acids may be fragmented or sheared to desired length, using a variety of mechanical, chemical and/or enzymatic methods. Nucleic acid may be sheared by sonication, brief exposure to a DNase, RNase, hydroshear instrument, one or more restriction enzymes, transposase or nicking enzyme, exposure to heat plus magnesium, or other methods. RNA may be converted to cDNA, e.g., before or after fragmentation. In one embodiment, nucleic acid from a biological sample is fragmented by sonication.

A biological sample as described herein may be lysed, homogenized, or fractionated in the presence of a detergent or surfactant. The concentration of the detergent in the buffer may be about 0.05% to about 10.0%, e.g., 0.1% to about 2%. The detergent, particularly a mild one that is non-denaturing, can act to solubilize the sample. Detergents may be ionic (e.g., deoxycholate, sodium dodecyl sulfate (SDS), N-lauroylsarcosine, and cetyltrimethylammonium bromide) or nonionic (e.g., octyl glucoside, polyoxyethylene(9)dodecyl ether, digitonin, polysorbate 80 such as that sold under the trademark TWEEN by Uniqema Americas (Paterson, N.J.), (C₁₄H₂₂O(C₂H₄)ₙ) sold under the trademark TRITON X-100 by Dow Chemical Company (Midland, Mich.), polidocanol, n-dodecyl beta-D-maltoside (DDM), or NP-40 nonylphenyl polyethylene glycol). A zwitterionic reagent may also be used in the purification schemes, such as zwitterion 3-14 and 3-[(3-cholamidopropyl)dimethyl-ammonio]-1-propanesulfonate (CHAPS). Urea may also be added. Lysis or homogenization solutions may further contain other agents, such as reducing agents. Examples of such reducing agents include dithiothreitol (DTT), β-mercaptoethanol, dithioerythritol (DTE), glutathione (GSH), cysteine, cysteamine, tricarboxyethyl phosphine (TCEP), or salts of sulfurous acid.

### Optical Mapping

FIG. 1 shows a method of optical mapping. From the nucleic acid sample, a plurality of single molecule optical maps are created. Optical mapping is a single-molecule technique for production of ordered restriction maps from individual molecules of nucleic acid. See Samad, et al., 1995, Optical Mapping: A Novel, Single-molecule Approach to Genomic Analysis, Genome Res. 5:1-4. During some applications, individual fluorescently labeled DNA molecules are elongated and fixed on the surface using methods of the invention. The added endonuclease cuts the DNA at specific points, and the fragments are imaged. Exemplary endonucleases include BglII, NcoI, XbaI, and BamHI. Exemplary combinations of restriction enzymes are shown in Table 1.

TABLE 1
Exemplary Combinations of Restriction Enzymes

(AflII ApaLI BglII), (AflII BglII NcoI), (ApaLI BglII NdeI), (AflII BglII MluI), (AflII BglII
PacI), (AflII MluI NdeI), (BglII NcoI NdeI), (AflII ApaLI MluI), (ApaLI BglII NcoI), (AflII
ApaLI BamHI), (BglII EcoRI NcoI), (BglII Ndel PacI), (BglII Bsu36I NcoI), (ApaLI BglII
XbaI), (ApaLI MluI NdeI), (ApaLI BamHI NdeI), (BglII NcoI XbaI), (BglII MluI NcoI), (BglII
NcoI PacI), (MluI NcoI NdeI), (BamHI NcoI NdeI), (BglII PacI XbaI), (MluI NdeI PacI),
(Bsu36I MluI NcoI), (ApaLI BglII NheI), (BamHI NdeI PacI), (BamHI Bsu36I NcoI), (BglII
NcoI PvuII), (BglII NcoI NheI), and (BglII NheI PacI)

Restriction maps can be constructed based on the number of fragments resulting from the digest. Generally, the final map is an average of fragment sizes derived from similar molecules.

Optical mapping and related methods are described in U.S. Pat. Nos. 5,405,519, 5,599,664, 6,150,089, 6,147,198, 5,720,928, 6,174,671, 6,294,136, 6,340,567, 6,448,012, 6,509,158, 6,610,256, and 6,713,263. All of these patents are incorporated herein by reference.

Optical Maps may be constructed as described in Reslewic et al., 2005, Whole-Genome Shotgun Optical Mapping of Rhodospirillum rubrum, Appl Environ Microbiol, 71 (9):5511-22.

Briefly, individual molecules from a sample are immobilized on a surface such as derivatized glass by virtue of electrostatic interactions between the negatively-charged DNA and the positively-charged surface. Each molecule is digested with one or more restriction endonuclease and stained with an intercalating dye such as the green fluorescent dye sold under the trademark YOYO-1 by Life Technologies (Carlsbad, Calif.). The fragments may be imaged by an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software. Each molecule immobilized on the surface thus produces a single molecule optical map.

The maps generated by the optical mapping can then be used to produce genome assemblies.

FIG. 2 gives a diagram for methods of producing genome assemblies from optical maps. First, the maps are generated as described above. These single molecule optical maps then are linked by similarity to create a plurality of groups of single molecule optical maps. Similarity linking is discussed in greater detail below. Similarity linking can involve making a table of links between pairs of maps (e.g., one link for every pair; at least one link for each map; links for some of the pairs or maps; etc.). In some embodiments, similarity is evaluated between pairs of maps and if a certain similarity metric is met for a pair, a link between that pair is created and recorded in a table. The single molecule optical maps can then be partitioned into groups based on the link table. Each of these groups contains single molecule optical maps that are similar to one another by some similarity metric. A plurality of contigs are generated for each of the groups.

The link-table partitioning process can be repeated for all of the contigs (in an optional step labeled “Iterate” in FIG. 2), or the contigs can be assembled into one or more genome maps. If the link-table partitioning is repeated, the contigs are partitioned into groups of contigs. For each group of contigs, the contigs within the group are assembled into a scaffold. The scaffolds can then be assembled into one or more genome maps. Preferably, steps of the methods are performed automatically using a computer system.

FIG. 3 depicts a system 129 for producing and analyzing optical maps. System 129 may generally include a bench computer system 104 coupled to wet work instruments. For example, system 104 (FIG. 3) may operate hardware that combines capillary flow technology for nucleic acid, e.g., DNA or RNA, deposition onto a surface with computer-controlled flow processing. For computer control, system 104 includes a processor 139 coupled to a memory 137 and input/output mechanisms 135. The capillary flow presents the nucleic acid molecules to a derivatized surface in long strands that are captured and held to the surface by electrostatic attraction. Once the nucleic acid molecules have been captured on the surface, reagents (e.g., washing solutions, buffers, enzymes, and nucleic acid stains), are flowed to and from the surface to produce restriction digests. The digests are subsequently imaged, thereby characterizing the nucleic acid molecule. The system may include a cartridge for characterizing a nucleic acid molecule, the cartridge including a reaction chamber having a derivatized bottom surface, at least one reagent reservoir, and a pump, in which the reaction chamber, the reagent reservoir, and the pump are fluidically connected to each other. The cartridge uses microfluidic components to link on-board reagent reservoirs via computer controlled valves and plumbing to a reaction chamber having a derivatized bottom surface. The derivatized bottom surface assists in elongating and fixing nucleic acid molecules, e.g., DNA or RNA, onto a surface so that the nucleic acid molecules remain accessible for enzymatic reactions. In certain embodiments, the derivatized bottom surface is derivatized glass.

The cartridge can be operably linked to bench system 104. Depending on the embodiment, the cartridge can further include at least one of the following: a reagent waste pad, a channel forming device configured to mate with the reaction chamber, a reaction chamber cap, or a heater/cooling device. The heater/cooling device can be located beneath the reaction chamber. In certain embodiments, the at least one reagent reservoir is a plurality of reservoirs, in which a first reservoir holds a TE wash reagent, a second reservoir holds a buffer, a third reservoir holds an enzyme, and a fourth reservoir holds a nucleic acid stain. Each reservoir can further include a loading port and a computer controlled valve for controlling flow of reagents from the reservoirs to the reaction chamber.

The cartridge is placed on the wet work instruments, reagents are loaded into the cartridge, using the loading ports associated with each reservoir. Loading can be accomplished by using any commercially available pipette. Once the reagents have been loaded, the orientation of the cartridge is adjusted to optimize flow of reagents within the cartridge. The cartridge can be placed flat (0° angle) on the surface of the preparation station. Alternatively, the cartridge can be oriented 90° to the surface of the preparation station. Generally, the cartridge can be oriented from about a 0° angle to about a 180° angle with respect to the surface of the preparation station. In a particular embodiment, the cartridge is tilted to a 60° angle with respect the surface of the preparation station in order to optimize reagent flow within the cartridge.

Once the cartridge has been oriented at the optimally determined angle for reagent flow, the preparation station (e.g., under the control of bench system 104) activates the pump in the cartridge and reagents are moved to the reaction chamber from the reservoirs and then aspirated from the reaction chamber to the reagent waste pad. The preparation station controls reagents exchange in the reaction chamber, flow rates, and temperature of the reaction chamber as required to complete washing, enzymatic digestion, and staining of the nucleic acid molecules for generation of restriction digests of the nucleic acid molecules. Further, flow is controlled (e.g., slow flow rates and controlled volumes) such that the nucleic acid molecules are not dislodged from the bottom surface of the reaction chamber.

Once the automated process is completed, the loading ports and any vent holes in the cartridge are sealed e.g., with adhesive tape or labels, and the cartridge is ready for readout on a imaging device, such as a fluorescing microscope operably linked via the computer system to a monitor or data storage. System 129 can thereby identify or measure each single molecule restriction map. Exemplary systems for optical mapping are discussed in U.S. Pub. 2013/0029323 to Briska, the contents of which are incorporated by reference.

The computer system 129 optionally includes modules for visualization, editing, or analyzing optical maps, which can be provided by, for example, a computer device 105 or a server 133 operating over a network 131. In certain embodiments, the system includes software modules, a database, or a combination thereof that provide similarity metrics, grouping, storage, assembly, and scaffolding as described herein. Preferably, the system provides contig linking and branched-path visualizations for heterozygous study samples. The system may additionally provide multi-tracked display of single molecule optical map data alongside external genomic data such as genes, sequence coverage, STS markers, SNP sites, CpG islands, chromosome banding, GC content, amino acid sequences of the encoded proteins, primary and tertiary structures of the encoded proteins, and molecules or agents that potentially interact with the DNA molecules or the encoded proteins, and other data collected from one or more external databases as indicated further infra.

Server 133 including memory 147 coupled to processor 149 and input/output connections 145 may include a database 151 that includes records 155 such as one or more of a flat file, a relational database, an object database or a data warehouse. A suitable relational database server for the system is, e.g., MySQL. Other examples of object databases that may be used include JYD Object Database or Objectivity/DB by Objectivity Inc. (Sunnyvale, Calif.). The database may be a data warehouse or a distributed database deployed over a network.

The system may include visualization and editing tools such as additional connectors that link the system to additional databases. These additional databases may also store information on single molecules and other biomedical information. These databases may be external databases such as those accessible over the Internet, e.g., GENBANK, SWIS-PROT, OMIM, and the NCBI SNP Database. The computer visualization and editing system can provide for visualization and editing of restriction maps as well as validation of these maps with sequence data retrievable from the connected databases.

In certain embodiments, computer device 105 provides a user interface (e.g., via input/output mechanism 135) that is capable of displaying single molecule fragments. A user may view the prior alignment and assembly of single molecules or fragments and, if necessary, minimally edit these data by removing, from contig assemblies by simple selection and keystroke of the delete key, whole maps with a high degree of error. Memory 137 coupled to processor 139 may perform steps described herein. Exemplary systems are described in U.S. Pat. No. 8,271,251 to Schwartz; U.S. Pub. 2013/0045879 to Mishra; and U.S. Pub. 2012/0254715 to Schwartz, the contents of each of which are incorporated by reference.

### Similarity Linking

In accordance with the invention, the single molecule optical maps that are created from the whole genome nucleic acid sample are separated or segregated on a similarity basis. Any suitable method of evaluating similarity among the single molecule optical maps may be performed. In some embodiments, a pairwise comparison is made between pairs of the maps. An exhaustive set of pairwise comparisons may be made (e.g., between each possible pair), or fewer pairwise comparisons can be made. For example, in some embodiments, once a pair of maps is compared and meets or satisfies a pre-determined similarity metric, at least one of those two maps is not subject to any more pairwise comparisons (e.g., get it and forget it mode).

Any suitable similarity metric can be used such as, for example, a scalar measure of molecule size, largest fragment size, smallest internal fragment size, number of cut sites, number of cut sites per length, others, or a combination thereof. A similarity metric can be based on a value calculated for each molecule or each single molecule map with a matrix of differences between values representing the similarity between each pair of molecules or each pair of single molecule maps. A similarity metric can include reference to biological information such as identity of restriction enzyme or genomic contents. For example, a similarity metric can include information about a ratio of number of restriction sites from one or more certain restriction enzymes to a number of restriction sites from one or more certain restriction enzymes (e.g., AflII cut sites per MluI cut sites). Other values that may factor into a similarity metric include GC content; phylogenetic distance; largest, shortest, mean, or other measure of fragment size; alignment score (e.g., from a heuristic alignment); barcodes or markers from sample (e.g., probe hybridization to surface-bound molecule); dye intensity; in silico RFLP; signature restriction patterns within the single molecule optical maps; other properties; or a combination thereof.

In certain embodiments, single molecule optical maps are evaluated for a similarity metric by approximating distribution counts of compared pairs and computing the probability of a match according to the Chen-Stein method. The Chen-Stein method approximates the distribution of occurrences of dependent events by the Poisson distribution. See Tang and Waterman, 2001, Local Matching of Random Restriction Maps, J Appl Prob 38:335-356; U.S. Pat. No. 6,340,567 to Schwartz; and U.S. Pub. 2005/0064406 to Zabarovsky, the contents of each of which are incorporated by reference.

In some embodiments, maps are evaluated for similarity by a series of alignments. The single molecule optical maps can be aligned to one another in a pairwise fashion. Alternatively, they may be aligned a reference. This can include generating in silico restriction maps derived from one or more chromosome of a reference genome of the organism, and aligning the single molecule restriction maps to the in silico references maps to thereby segregate the single molecule restriction maps into a plurality of groups.

Map alignments (e.g., pairwise alignments between the single molecule optical maps, or alignments between the single molecule optical maps and the in silico chromosome maps of the reference) can be generated with a dynamic programming algorithm that finds the optimal alignment of two maps according to a scoring model that incorporates fragment sizing errors, false and missing cuts, and missing small fragments. See e.g., Myers and Huang, 1992, An O(N² log N) restriction map comparison and search algorithm, Bull Math Biol 54(4):599-618 and Waterman et al., 1984, Algorithms for Restriction Map Comparisons, Nucleic Acids Res 12(1 Pt 1):237-242. For a given alignment, the score is proportional to the log of the length of the alignment, penalized by the differences between the two maps, such that longer, better-matching alignments will have higher scores. From these alignments, a pair-wise alignment analysis can be performed to determine “percent dissimilarity” between the pairs of single molecule optical maps, taking the total length of the unmatched regions in both maps divided by the total size of both maps.

Once the similarities between maps are evaluated, maps can be linked that satisfy a threshold similarity metric. This can be used to group the maps. The optical maps from each group can be assembled, e.g., in a de novo manner, into contigs.

The above described process may optionally be repeated, in an iterative fashion, for the contigs. That is, the contigs can be fed to the similarity algorithm. Similarities between the contigs can be evaluated and similar contigs can be grouped into groups. Within each group, the contigs can be assembled into scaffolds. Then, the scaffolds can be assembled into genomic assemblies. Where the iterative step is not performed, the genomic assembly can be provided by the initial assembly of the contigs.

The assembly into a genomic assembly can proceed by any suitable algorithm. For example, assembly can be a de novo assembly using the dissimilarity scores from a pairwise alignment step. The dissimilarity measurements are used as inputs into the agglomerative clustering method “Agnes” as implemented in the statistical package “R”. Briefly, this clustering method works by initially placing each entry in its own space, then iteratively joining the single molecule optical map to the optical map of the reference contig that most closely matches that single molecule optical map, thereby producing contigs within each bin.

In some embodiments, assembly proceeds by a computer program that implements the algorithm known as Gentig. Gentig uses an approximation algorithm for finding an almost optimal scoring set of contigs, while constraining the false positive error rate below a negligible value. Under a simple overlap rule, dubbed Type D, that determines when two genomic DNA molecules can be deemed to have a common sub-fragment, a conservative estimate of the false probability can be given as

$$\frac{4}{{p}_{c}^{4}} exp ( - β n / 2 ) {\sum }_{i = k}^{∞} \frac{{( β n / 2 )}^{i}}{i !}$$

where p_{c} is the digestion rate, B is the relative sizing error, n is the expected number of restriction fragments per genomic DNA molecule, and k is the integer parameter directly related to overlap threshold ratio theta. See Lin, et al., 1999, Whole-Genome Shotgun Optical Mapping of Deinococcus radiodurans, Science 285:1558-1562; U.S. Pat. No. 7,831,392 to Antoniotti; U.S. Pub. 2013/0045879 to Mishra; and U.S. Pub. 2003/0087280 to Schwartz, the contents of each of which are incorporated by reference.

Other methods of assembly may be used including assembling contigs (or scaffolds) by aligning the contigs to a reference genome or performing an exhaustive pairwise alignment among the contigs (or scaffolds). Assembly of optical maps is discussed in U.S. Pub. 2013/0029877 to Dykes; U.S. Pub. 2012/0183953 to Xiao; and U.S. Pub. 2007/0148674 to Berres, the contents of each of which are incorporated by reference.

It is also reported that methods of the invention provide advantageous benefits in the analysis of heterozygous samples. Grouping of single molecule optical maps by similarity will group maps from similar chromosomes into the same group. Thus, for example, in some embodiments, where a sample includes a mixture of heterozygous diploid chromosomes, the methods will produce a group for each chromosome. Within each group, maps will be included that represent both of the diploid heterozygous haplotypes. The visualization system described above can be used for assembly of the maps within the heterozygous group. Assembly can include linking the contigs together to form a branched path.

FIG. 4 illustrates a branched path assembly according to certain embodiments. The branched path represents the heterozygous nature of the sample. A converged section of the path represents regions of similarity between the paired sources chromosomes, while a divergent, split section indicates dissimilarity.

While discussed in the immediately preceding paragraph in terms of heterozygous diploid chromosomes, branched path assembly and visualization has applications in other analyses involving unlike chromosomes. For example, path branching can represent non-stoichiometric heterozygosity, and may be used to analyze allele frequencies other than 0%, 50%, and 100%. For example, where a sample has one allele present as a minority, branched path assembly can represent the presence of that allele therefore giving a true negative test for loss of heterozygosity. Thus the assembler system can explicitly represent the heterozygous nature of the sample.

As described above, the invention provides systems and methods for generating sets of contigs of single molecule optical maps from a sample containing nucleic acid. The sets of contigs may be used for comparative genomics analysis to identify structural variations, including intra- and inter-chromosomal rearrangements. For example, the assembled contigs can be analyzed using any of a variety of comparative genomics analysis techniques to reveal structural variations, including intra- and inter-chromosomal rearrangements. Comparative genomic analysis using optical maps is shown for example in Zhou et al., 2004, Single-Molecule Approach to Bacterial Genomic Comparisons via Optical Mapping, J Bacteriol, 186(22):7773-7782, the contents of which are incorporated by reference herein in its entirety.

Implemented by Computer(s)

The methods disclosed herein are capable of being carried out by one or more general-purpose computers that are programmed by one or more software applications. And, in particular, it is noted that the processing depicted in FIG. 1 can be carried out by such one or more computers. Each of the one or more programmed computers will include at least one processor and one or more types of storage such as RAM, ROM, hard disk, optical disk, etc. The computer(s) typically also will have locally and/or will have access to remotely one or more databases containing data representative of the optical maps, DNA information, and other real-world chemical and/or biological elements, compounds, etc. that are disclosed herein. These data are processed by the programmed computer(s) to perform the steps and accomplish the methods disclosed herein.

FIG. 3 shows system 129 according to some embodiments. Device 105 may include a PC, tablet, or any other suitable device. Device 105 will generally include a memory 137 coupled to a processor 139 as well as an input/output mechanism 135. Server 321 may include one or more computer devices using one or more of processor 149 coupled to memory 147 as well as input/output device 145. Memory 137 or may be taken to include one or more of a volatile or persistent memory such as RAM or storage. Computer storage can be provided by a hard drive (e.g., magnetic disk drive), flash memory, solid state drive (SSD), removable compact flash card, or similar. Processor 139 or 149 will generally include one or more computer processor such as a microchip made by Intel. Input/output 135 or 145 may include a mouse, keyboard, monitor, screen, touchscreen, network interface card, Wi-Fi card, cellular modem, Ethernet jack, USB jack, radio-frequency identification transponder, similar, or combinations thereof. Device 105 may be in communication with server 133 via network 131. Network 131 may include one or more of a wireless or wired internet communication device (e.g., router, hub, or switch), cellular tower, modem, land lines, satellites, antennae, similar structures, or a combinations thereof. Server 321 may house a database 151 that includes records 155 wherein may be stored any of the information described or required herein. Any such information may additionally or alternatively be stored in memory 137. Preferably, memory 137 or memory 147 is a tangible, non-transitory device that may contain the instructions executable to cause a system or device of the method to perform any of the steps, methods, or functions described herein. Exemplary computer systems for implementing methods disclosed herein are discussed in U.S. Pub. 2012/0249580 to Schwartz. As used herein, the word “or” means “and or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of obtaining genomic information, the method comprising:
generating a plurality of single molecule optical maps from a nucleic acid sample;
linking pairs of the maps according to a similarity metric;
dividing the plurality of maps into groups comprising linked maps;
assembling the linked maps within each group into a contig to produce a plurality of contigs; and
assembling the plurality of contigs into at least one genome assembly.

2. The method of claim 1, wherein assembling the plurality of contigs comprises:
linking pairs of the contigs according to a contig similarity metric;
dividing the contigs into contig groups comprising linked contigs;
assembling the linked contigs within each contig group into a scaffold to produce a plurality of scaffolds; and
assembling the plurality of scaffolds into the at least one genome assembly.

3. The method of claim 1, further comprising assembling the plurality of contigs into two or more genome assemblies.

4. The method of claim 1, wherein assembling the plurality of contigs into the at least one genome assembly comprises linking the plurality of contigs together to form a branched path.

5. The method of claim 4, wherein the branched path comprises at least one converged section and at least one diverged section.

6. The method of claim 5, wherein the at least one diverged section represents heterozygosity in the sample.

7. The method of claim 6, wherein the at least one genome assembly represents a diploid genome that is heterozygous at the at least one diverged section.

8. The method of claim 1, wherein generating a single molecule optical map comprises:
introducing nucleic acid from the sample to a charged substrate so that the nucleic acids become elongated and fixed on the substrate in a manner in which the nucleic acids remain accessible for enzymatic reactions;
digesting the nucleic acids enzymatically to produce one or more restriction digests; and
constructing a map from the restriction digests.

9. The method of claim 8, wherein the substrate is derivatized glass.

10. The method of claim 1, wherein the sample comprises human tissue or fluid.

11. The method of claim 1, wherein assembling comprises determining contig arrangement.

12. The method of claim 1, wherein the similarity metric comprises a measure of a number of cutting sites per length of nucleic acid molecule for a restriction enzyme.

13. The method of claim 1, wherein the similarity metric comprises an alignment score.

14. The method of claim 1, further comprising:
generating an in silico ordered restriction map from a reference genome; and
obtaining the similarity metric by comparing the maps to the in silico ordered restriction.

15. The method of claim 1, further comprising:
using a computer system to perform the recited steps, wherein the computer system comprises a processor and a non transitory memory.

16. A method of obtaining contigs, the method comprising:
generating a plurality of single-molecule ordered restriction maps from a nucleic acid sample;
segregating the maps into a plurality of groups, each group comprising a plurality of linked maps linked by a similarity metric; and
assembling the linked maps within each group into a contig to produce a plurality of contigs.

17. The method of claim 16, further comprising:
linking pairs of the contigs that satisfy a contig similarity metric;
segregating the contigs into contig groups comprising linked contigs; and
assembling the linked contigs within each contig group into a scaffold to produce a plurality of scaffolds.

18. The method of claim 17, further comprising assembling the plurality of scaffolds into the at least one genome assembly.

19. The method of claim 16, wherein the similarity metric comprises a measure of a number of cutting sites per length of nucleic acid molecule for a restriction enzyme.

20. The method of claim 16, wherein the similarity metric comprises an alignment score.

21. The method of claim 16, further comprising:
generating an in silico ordered restriction map from a reference genome; and
obtaining the similarity metric by comparing the maps to the in silico ordered restriction

22. The method of claim 16, further comprising assembling the plurality of contigs into at least one genome assembly.

23. The method of claim 22, wherein assembling the plurality of contigs into the at least one genome assembly comprises linking the plurality of contigs together to form a branched path.

24. The method of claim 23, wherein the branched path comprises at least one converged section and at least one diverged section.

25. The method of claim 24, wherein the at least one diverged section represents heterozygosity in the sample.
